(19) United States
(12) Patent Application Publication
(10) Pub. No.: US 2026/0183547 A1
(43) Pub. Date: Jul. 2, 2026

(54) SYSTEM FOR CONTROL OF SPASTICITY

(71) Applicant: ECOLE POLYTECHNIQUE FÉDÉRALE DE LAUSANNE, Lausanne (CH)

(72) Inventors: Salif KOMI, COPENHAGUE (DK); Robin DEMESMAEKER, Morges (CH); Karen MINASSIAN, Vienna (AT); Fabien WAGNER, Pully (CH); Grègoire COURTINE, Lausanne (CH); Jocelyne BLOCH, PAUDEX (CH)

(21) Appl. No.: 19/130,528

(22) PCT Filed: Nov. 17, 2023
(86) PCT No.: PCT/EP2023/082158

§ 371 Date: May 15, 2025

(30) Foreign Application Priority Data
Nov. 21, 2022 (EP) 22208454.3

(51) Int. Cl. A61N 1/36

(52) U.S. Cl. CPC A61N 1/36067; A61N 1/36062; A61N 1/36139; A61N 1/36171; A61N 1/36175; A61N 1/36178

(57) ABSTRACT

The present invention relates to a neuromodulation/neurostimulation system (10) for the treatment of spasticity in a mammal, said system (10) comprising: —at least one control unit (12) configured and arranged to provide stimulation data, and—at least one stimulation unit (14), operatively connected to the at least one control unit (12), said at least one stimulation unit (14) being configured and arranged to deliver epidural electrical stimulation to the spinal cord of said mammal, according to said stimulation data, wherein the at least one stimulation unit (14) includes a biocompatible implantable lead (18) configured and arranged to cover at least a portion of the spinal cord of said mammal to deliver epidural electrical stimulation to the dorsal roots innervating the spastic muscles to target neuronal circuitry responsible for spastic episodes.

### CROSS REFERENCE TO RELATED APPLICATIONS

The present application is a U.S. National Phase of International Application No. PCT/EP2023/082158 entitled “SYSTEM FOR CONTROL OF SPASTICITY,” and filed on Nov. 17, 2023. International Application No. PCT/EP2023/082158 claims priority to European Patent Application No. 22208454.3 filed on Nov. 21, 2022. The entire contents of each of the above-listed applications are hereby incorporated by reference for all purposes.

### TECHNICAL FIELD

The present invention belongs to the technical field of spinal cord stimulation for alleviating or preventing effects of spinal cord injury (SCI) and/or other neurological disorders in a mammal, in particular a human.

### BACKGROUND AND SUMMARY

More specifically, the present invention relates to a neuromodulation/neurostimulation system specifically targeted at mitigating or preventing spastic episodes in a mammal.

In particular, said mammal may be a human with SCI and/or other neurological disorders such as a stroke, multiple sclerosis, cerebral palsy or cancer of the neurological tissue which impair operation of descending neurological pathways.

The spinal cord is an integral part of the central nervous system (CNS). SCI may result, among others, in motor, sensory and autonomic deficits.

Spasticity may follow as a consequence of SCI and/or other neurological disorders.

Spasticity is a clinical condition determined by an abnormal increase in muscle tone or stiffness of muscle, which may interfere with movement and/or speech, or otherwise generate discomfort or pain in a subject.

Spasticity leads to disability and reduced quality of life, and is thus ranked among the top healthcare priorities by subjects affected by SCI and/or other neurological disorders.

Methods and systems for alleviating spasticity are known in the art.

In particular, a known clinical approach for alleviating the symptoms of spasticity includes the administration of pharmacological drugs.

For instance, antispastic agents acting at the spinal level such as Baclofen are commonly used as a pharmacological remedy for mitigating spasticity of voluntary muscle resulting from SCI and/or other disorders such as multiple sclerosis, tumours of the spinal cord, syringomyelia, motor neurone disease, transverse myelitis or traumatic partial section of the cord.

WO2020/141141A1 discloses pharmaceutical compositions for the treatment of a disease or disorder selected from spasticity due to multiple sclerosis, spinal cord injury or cerebral palsy, and alcoholism, said compositions comprising cocrystals of (R)-Baclofen, in particular cocrystals of (R)-Baclofen with cinnamic acid, benzoic acid, salicylic acid and ferulic acid.

US2020/078327A1 discloses Baclofen solutions for use in the treatment of spasticity, brain injury, cerebral palsy, spinal cord injury, cervical injury, multiple sclerosis, thoracic injury, or withdrawal symptoms, said solutions comprising greater than 2 mg/mL Baclofen and further including between 5 mM and 25 mM of a phosphate or sulfate species.

US2014/128355A1 discloses a solution for delivery to patients suffering from spasticity comprising a high concentration of Baclofen (10 mg/ml) in a multivalent physiological ion solution such as artificial cerebrospinal fluid.

WO2008/003093A1 discloses pharmaceutical compositions for reducing muscle spasticity comprising at least one alpha2-adrenergic agonist or Baclofen and at least one alpha1-adrenergic agonist.

Known pharmacological approaches for alleviating spasticity are not limited to Baclofen-based compounds.

US2021/130285A1 discloses pharmaceutically active compounds that show utility in the treatment of central nervous system (CNS) diseases and disorders, such as anxiety, depression, insomnia, migraine headaches, schizophrenia, neurodegenerative diseases (e.g., Parkinson's disease, Alzheimer's, ALS, and Huntington's disease), spasticity, and bipolar disorders.

EP3658177A1 discloses a composition for use in the treatment of lower limb spasticity in a patient, in particular an 18-years-old or under patient, comprising botulinum neurotoxin.

WO2015/085118A1 discloses a method of treating spasticity in a human subject comprising intrathecally administering (e.g., through intrathecal pump) a therapeutically effective amount of dantrolene or a pharmaceutically acceptable salt thereof.

Further solutions for mitigating spasticity rely on the use of compositions including muscle-derived progenitor cells (MDCs).

Uses of MDCs for the treatment of cosmetic or functional conditions, including skeletal muscle weakness, muscular dystrophy, muscle atrophy, spasticity, myoclonus and myalgia are disclosed, e.g., in US2021145892A1.

Pharmacological remedies to spasticity allow obtaining a sustained suppression of spastic episodes.

However, such remedies have the disadvantage that they cannot be turned on/off in an immediate situation-specific fashion.

Further, pharmacological remedies cannot be targeted at reducing spastic contractions in specific muscle groups while not influencing others. As a consequence, unwanted side effects on the normal muscular functioning may occur.

In addition, Baclofen-based antispastic agents may determine side effects such as somnolence, dry mouth, respiratory depression, light-headedness, fatigue, confusional state, dizziness, headache, insomnia, depression, euphoric mood, myalgia, muscular weakness, ataxia, tremor, nystagmus, hallucination, nightmare, or even paraesthesia, dysarthria and dysgeusia, which may dramatically affect life quality of a subject.

Another known approach for alleviating severity of spastic episodes relies on magnetic stimulation of the spinal nerve.

US2020/316396A1 discloses methods, devices and systems for the treatment of spasticity by using low-frequency repetitive magnetic fields to enhance communication in the spinal nerve, thereby allowing improved relaxation, control, and coordination in a muscle.

Yet another known approach for spastic episodes mitigation includes providing continuous neuromodulation of the sacral spinal nerve roots, in particular through Transcutaneous Electrical Nerve Stimulation (TENS).

US2002/019650A1 discloses a method of controlling spasticity in patients having SCI including applying electrical stimuli to the sensory and motor sacral nerve pathways at one or more of the S1 to S4 levels through electrodes that are connected directly to the respective nerve roots. Electrical stimulation is applied generally by means of invasive electrodes maintained in the proximity of the spinal nerve roots. Stimulation may be delivered with stimulation frequency of less than 20 pulses/second, preferably about 10 to 15 pulses/second.

Although effective, however, the continuous neuromodulation of the sacral spinal nerve roots still shows some disadvantages.

In particular, similar to the pharmacological approach described above, continuous neuromodulation of the sacral spinal nerve roots cannot be targeted at reducing spastic contractions in specific muscle groups while not influencing others, which may lead to unwanted side effects on normal muscular functioning.

Neurostimulation techniques, in particular epidural electrical stimulation (EES), are known in the art.

Specifically, said known techniques rely on the use of one or more patterns of electrical stimulation that are applied to the nervous system of a subject.

Neurostimulation is widely used for improving/restoring motor or autonomic functions in subjects with SCI and/or other neurological disorders.

Experimental results on the use of neurostimulation for improving/restoring motor functions in subjects affected by SCI and/or other neurological disorders can be found, inter alia, in:
Wagner, F. B. et Al., Targeted neurotechnology restores walking in humans with spinal cord injury, Nature 563, 65-71. https://doi.org/10.1038/s41586-018-0649-2);
Capogrosso, M. et Al., Configuration of electrical spinal cord stimulation through real-time processing of gait kinematics, Nat Protoc 13, 2031-2061, https://doi.org/10.1038/s41596-018-0030-9, and
Rowald, A. et Al., Activity-dependent spinal cord neuromodulation rapidly restores trunk and leg motor functions after complete paralysis, Nature Medicine, 28 (2), 260-271. https://doi.org/10.1038/s41591-021-01663-5.

Neurostimulation is commonly used to mitigate consequences of neuromotor disorders after SCI, ischemic injury resulting from a stroke, or a neurodegenerative disease such as Parkinson's disease, which may affect cardio-vascular, bladder, bowel, and sexual functions.

US2019344075A1 discloses a system for spatiotemporal electrical neurostimulation of the spinal cord of a subject, the system comprising a signal processing device that receives signals from the subject and operates signal-processing algorithms to elaborate stimulation parameter settings; one or more multi-electrode arrays suitable to cover at least a portion of said subject for applying to said subject a selective spatiotemporal stimulation of the spinal circuits and/or dorsal roots, and an Implantable Pulse Generator (IPG) configured to receive stimulation parameter settings from said signal processing device and simultaneously deliver independent current or voltage pulses to the one or more multiple electrode arrays to provide a multipolar stimulation.

WO2019110401A1 discloses a system for planning and/or providing neurostimulation for a patient, the system comprising a pathological spinal cord map storage module for storing at least one pathological spinal cord map describing the activation of the spinal cord of a patient; a healthy spinal cord map storage module for storing at least one reference map describing physiological activation of the spinal cord of at least one healthy subject; an analysis module configured and arranged such that the pathological spinal cord map and the reference map can be compared and/or analyzed automatically to generate a deviation map describing the difference between the pathological spinal cord map and the reference map; and a compensation module which is configured and arranged to calculate, on the basis of the deviation map, a neurostimulation protocol for compensating the activation difference between the pathological spinal cord map and the reference map. Studies have also been carried out on the potential use of SCS in mitigating severity of spastic episodes.

In particular, a pilot study has been implemented on three subjects with chronic motor-incomplete SCI who could walk ≥10 m to examine the effects of transcutaneous spinal cord stimulation (tSCS) on lower-limb spasticity. Two interconnected stimulating skin electrodes (Ø 5 cm) have been placed paraspinally at the T11/T12 vertebral level, and two rectangular electrodes (8×13 cm) have been placed on the abdomen for reference. Biphasic 2 ms-width pulses were delivered at 50 Hz for 30 minutes at intensities that allowed producing paraesthesias, but no motor responses in the lower limbs. As a result, the index of spasticity derived from the pendulum test changed from 0.8±0.4 pre- to 0.9±0.3 post-stimulation, with an improvement in the subject with the lowest pre-stimulation index. Exaggerated reflex responsiveness was decreased after tSCS across all subjects, with the most profound effect on passive lower-limb movement (pre- to post-tSCS EMG ratio: 0.2±0.1), as was non-functional co-activation during voluntary movement (Hofstoetter U.S. et Al., Modification of spasticity by transcutaneous spinal cord stimulation in individuals with incomplete spinal cord injury, J Spinal Cord Med, 2014; 37 (2): 202-211 doi: 10.1179/2045772313Y.0000000149).

Transcutaneous spinal cord stimulation also showed some drawbacks in resulting sometimes unstable over time, further than being significantly dependent on skin-electrode attachment. Furthermore, transcutaneous stimulation requires the operator to place the electrodes on the patient's body every time stimulation is to be delivered, which may be inconvenient.

Recent literature has underlined that SCS, a once promising therapy for mitigating the effects of spasticity, has largely been relegated to a permanent experimental status. SCS has been tested in over 25 different conditions starting from when a potentially beneficial effect was first reported in 1973. However, the lack of a fully formed understanding of the pathophysiology of spasticity, archaic study methodology, and the early technological limitations of implantable hardware has limited the validity of many studies. SCS has the advantage of offering a measure of control for spasticity that cannot be duplicated with other interventions (Nagel S. J. et Al., Spinal cord stimulation for spasticity: historical approaches, current status, and future directions, Neuromodulation, 2017; 20:307-321).

Another limit of the above-mentioned experiments and studies is that they all relied on application of spatially and temporally unspecific stimulation, while spasticity is a sporadic and spatially restricted phenomenon.

Thus, there is the need to provide a different and more effective clinical approach, tailored to target specific neuronal circuitry responsible for the current status and location of the spastic activity.

In addition, there is also the need to provide a targeted approach to spasticity reduction that can leverage the recent progresses in the development of electrode interfaces and active neurostimulators with real-time capacities (Nagel et Al., 2017, Neurotrauma).

This object is achieved by the provision of a neuromodulation/neurostimulation system as described herein.

In particular, the present invention provides a neuromodulation/neurostimulation system for the treatment of spasticity in a mammal.

Specifically, said mammal may be a human affected by SCI and/or other neurological disorders such as a stroke, multiple sclerosis, cerebral palsy or cancer of neurological tissue which impair operation of descending neurological pathways that normally facilitate motor control.

The system comprises at least one control unit.

In one embodiment, the system may comprise a single control unit.

The at least one control unit is configured and arranged to provide stimulation data.

The system further comprises at least one stimulation unit.

In one embodiment, the system may comprise a single stimulation unit.

The at least one stimulation unit is operatively connected to the at least one control unit.

The at least one stimulation unit is configured and arranged to deliver epidural electrical stimulation (EES) to the spinal cord of said mammal, according to said stimulation data.

The at least one stimulation unit includes a biocompatible implantable lead.

The implantable lead is configured and arranged to cover at least a portion of the spinal cord of said mammal, in particular a human, to deliver EES to the dorsal roots innervating the spastic muscles to target neuronal circuitry responsible for spastic episodes.

The invention is based on the basic idea that a stimulation system has to be provided which specifically targets and modulates neural circuitry responsible for the current status and location of spastic activity in a way that enables to effectively mitigate or prevent spastic muscle contractions in one or more specific muscle groups while not influencing the others, thereby preventing the occurrence of unwanted side effects on normal muscular functioning.

Furthermore, spatially and temporally targeted anti-spastic stimulation can be effectively combined with delivery of different kinds of electrical stimulation, such as neuromodulation therapies directed at restoring/improving motor functions or the like.

Advantageously, the implantable lead may include one or more biocompatible implantable multi-electrode arrays.

The at least one stimulation unit may include an Implantable Pulse Generator (IPG).

Advantageously, the implantable lead may be configured and arranged to deliver EES to the spinal cord of said mammal, in particular a human, more specifically to the dorsal roots of the spinal cord, at spinal cord level responsible for the lower limbs, preferably at level T11/T12-L3 (vertebrae) (or looking at spinal cord itself the section of the spinal cord and its dorsal roots for T12 to S2) (cf. also FIG. 3).

Additionally or alternatively, the implantable lead may be configured and arranged to deliver EES to the spinal cord of said mammal, in particular a human, at spinal cord level responsible for the upper limbs, preferably over the cervical region of the spinal cord.

Additionally or alternatively, the implantable lead may be configured and arranged to deliver EES to the spinal cord of said mammal, in particular a human, at spinal cord level responsible for the trunk muscles, in particular over the thoracic region of the spinal cord.

In one embodiment, the at least one control unit may be configured and arranged to control the at least one stimulation unit to deliver continuous EES to the spinal cord of said mammal.

In this embodiment, the system operates as an open-loop system.

It has been found that, by providing continuous EES to targeted locations on the spinal cord of said mammal, in particular a human, frequency of spastic events and severity of the spasms can be effectively reduced.

The neuromodulation/neurostimulation system may further comprise a detection unit.

In one embodiment, the detection unit is configured and arranged to detect onset of spastic muscle activity in said mammal, in particular a human.

In this embodiment, when onset of spastic muscle activity is detected, the detection unit is further configured and arranged to transmit an output signal to the at least one control unit.

In this embodiment, the at least one control unit is further configured to operate the at least one stimulation unit to provide EES to the spinal cord of said mammal in response to the received signal from the detection unit.

In this embodiment, the system operates as a closed-loop system.

It has been found that, by triggering targeted EES when onset of spastic muscle activity is detected, it is possible to effectively reduce severity of an ongoing spastic episode.

For example, stimulation can be delivered for a fixed amount of time or until the spastic episode is over.

Optionally, stimulation may be manually operated by the patient during an ongoing spastic episode, to reduce severity of spastic muscle activity.

In one embodiment, the detection unit is configured and arranged to detect onset of a spasticity-inducing activity and/or movement in said mammal, in particular a human, based on one or more preset parameters stored in a memory.

In this embodiment, when onset of a spasticity-inducing activity and/or movement is detected, the detection unit is further configured and arranged to transmit an output signal to the at least one control unit.

In this embodiment, the at least one control unit is further configured to operate the at least one stimulation unit to provide EES to the spinal cord of said mammal in response to the received signal from the detection unit, to prevent occurrence of a spastic episode.

In this embodiment, the system as well operates as a closed-loop system.

It has been found that, by triggering targeted EES before a subject performs an activity and/or movement which is identified as spasticity-inducing, it is possible to temporally prevent a spastic episode from occurring.

Advantageously, the detection unit may include at least one electromyography sensor unit.

Additionally or alternatively, the detection unit may include at least one inertial measurement unit.

The stimulation data may comprise at least frequency, amplitude, pulse width and/or anodic/cathodic configurations.

Preferably, stimulation is delivered with frequencies that are beyond frequencies leading to functional muscle activation.

Advantageously, the stimulation frequency is between 100 Hz and 1500 Hz.

Advantageously, the pulse width is between 60 μs and 1000 μs.

Preferably, the pulse width is of 300 μs.

Advantageously, stimulation is delivered with a stimulation amplitude that is around motor threshold level.

The at least one control unit may further comprise an oscillation control module.

In particular, the oscillation control module may be configured and arranged to provide an input between 0.01 Hz and 0.2 Hz low frequency oscillation in the amplitude and/or frequency.

Preferably, the oscillation control module may be configured and arranged to provide an input of 0.1 Hz low frequency oscillation in the amplitude and/or frequency.

The at least one stimulation unit may be configured and arranged to provide at least one burst train stimulation pulse.

Preferably, the at least one stimulation unit may be configured and arranged to provide at least one burst of several pulses.

More preferably, the at least one stimulation unit may be configured and arranged to provide at least one burst of 2 to 5 pulses.

The present invention further relates to the use of the neuromodulation/neurostimulation system described above for the treatment of spasticity in a mammal, preferably a human, after SCI and/or other neurological disorders.

The neuromodulation/neurostimulation system of the invention may be used in a method for treatment of spasticity in a mammal.

In particular, said mammal may be a human affected by SCI and/or other neurological disorders such as a stroke, multiple sclerosis, cerebral palsy or cancer of neurological tissue which impair operation of descending neurological pathways that normally facilitate motor control.

The method may include:
positioning the biocompatible implantable lead on the body of said mammal, in particular a human, to cover at least a portion of the spinal cord of said mammal, and deliver EES to the dorsal roots innervating the spastic muscles to target neuronal circuitry responsible for spastic episodes, and
controlling the at least one stimulation unit to deliver EES to the spinal cord of said mammal, in particular a human, according to stimulation data provided by the control unit.

In one example, the method may include delivering EES to the spinal cord of said mammal, in particular a human, at spinal cord level T12-S2.

In one example, the method may include delivering EES to the spinal cord of said mammal, in particular a human, at spinal cord level responsible for the upper limbs, preferably over the cervical region of the spinal cord.

In one example, the method may include delivering EES to the spinal cord of said mammal, in particular a human, at spinal cord level responsible for the trunk muscles, in particular over the thoracic region of the spinal cord. In one example, the method may include delivering continuous EES to the spinal cord of said mammal.

In this example, the method operates the system as an open-loop system.

In one example, the method may include:
detecting, through a detection unit, onset of spastic muscle activity in said mammal, in particular a human, based on one or more preset parameters stored in a memory;
transmitting an output signal to the at least one control unit when onset of spastic muscle activity is detected, and
operating the at least one stimulation unit to provide EES to the spinal cord of said mammal in response to the received signal from the detection unit.

In this example, the method operates the system as a closed-loop system.

In this example, stimulation can be delivered for a fixed amount of time or until the spastic episode is over.

Optionally, stimulation may be manually operated by the patient during an ongoing spastic episode, to reduce severity of spastic muscle activity.

In one example, the method may include:
detecting, through a detection unit, onset of a spasticity-inducing activity and/or movement in said mammal, in particular a human, based on one or more preset parameters stored in a memory;
transmitting an output signal to the at least one control unit when spasticity-inducing activity and/or movement is detected, and
operating the at least one stimulation unit to provide EES to the spinal cord of said mammal in response to the received signal from the detection unit.

In this example, the method as well operates the system as a closed-loop system.

Advantageously, the detection unit may include at least one electromyography sensor unit.

Additionally or alternatively, the detection unit may include at least one inertial measurement unit.

The method may include delivering EES with stimulation frequency between 100 Hz and 1500 Hz.

The method may include delivering EES with pulse width between 60 μs and 1000 μs.

Preferably, the method may include delivering EES with pulse width of 300 μs.

The method may include delivering EES with a stimulation amplitude that is around motor threshold level.

In one example the method may include providing, through an oscillation control module, an input between 0.01 Hz and 0.2 Hz low frequency oscillation in the amplitude and/or frequency.

Preferably, the method may include providing an input of 0.1 Hz low frequency oscillation in the amplitude and/or frequency.

The method may include providing at least one burst train stimulation pulse.

Preferably, the method may include providing at least one burst of several pulses.

More preferably, the method may include providing at least one burst of 2 to 5 pulses.

### BRIEF DESCRIPTION OF THE FIGURES

Further details and advantages of the present invention shall now be disclosed in connection with the drawings, where:

FIG. 1 is a schematic overview of a neuromodulation/neurostimulation system for the treatment of spasticity in a mammal, according to an embodiment of the present invention;

FIG. 2A is a diagram showing a condition where no stimulation is applied to a subject (e.g., a patient), and the subject shows frequent and severe spastic episodes;

FIG. 2B is a diagram showing an embodiment where continuous EES is delivered to a subject, showing a reduction in frequency and severity of spastic episodes;

FIG. 2C is a diagram showing an embodiment where EES is delivered to a subject upon detecting onset of a spasticity-inducing activity and/or movement in said subject, showing a mitigation in severity of spastic episodes;

FIG. 2D is a diagram showing an embodiment where EES is delivered to a subject upon detecting onset of spastic muscle activity in said subject, showing an immediate reduction in severity of an ongoing spastic episode;

FIG. 3 is a diagram showing a concept of targeted EES for the lower limbs of a subject. Leg muscles are innervated by the lumbosacral spinal cord in an individual-specific rostro-caudal spatial organization. An electrode array (also called paddle lead) is implanted on the lumbosacral spinal cord to stimulate specific spinal segments innervating respective specific muscles;

FIG. 4A and FIG. 4B is a diagram showing EES systems used in a clinical study involving different participants P1-P5. (a) System used with participants P1-P4: EES commands from a custom-built stimulation software are transmitted to the implantable pulse generator (IPG, Medtronic ActivaRC) via Bluetooth (1) to a module that converts them into infrared signals (2), which are then transferred to the stimulation programmer device (2′). The stimulation programmer transmits EES commands into the IPG via induction telemetry (4), using an antenna (3) taped to the skin and aligned to the IPG. EES is delivered through the paddle array (5). Participants P1-P2 were implanted with the Medtronic Specify 5-6-5 paddle lead, whereas P3-P4 were implanted with an investigational new paddle lead. This communication chain allows the control of up to 4 concomitant stimulation waveforms in real-time, with a response latency of approximately 120 ms. (b) System used with participant P5: EES programs are configured through the Medtronic clinician tablet and can be activated independently by the participant with the Medtronic patient programmer. Through Near-Field Magnetic Induction communication (NFMI), these devices wirelessly communicate with a Medtronic Intellis IPG implanted in the abdominal flank and connected to a Medtronic Specify 5-6-5 paddle array;

FIG. 5 is a diagram showing an example of a spasticity-inducing passive movement of a subject;

FIG. 6 is a diagram showing the effects of delivery of antispastic stimulation to the left leg of participant P2;

FIG. 7 is a diagram showing combined results of Modified Ashworth Scale (MAS) with and without stimulation for participants P1-P5;

FIG. 8 is a diagram showing the effects of turning on targeted EES during an ongoing spastic event, resulting in an immediate alleviation of spastic muscle activity.

### DETAILED DESCRIPTION

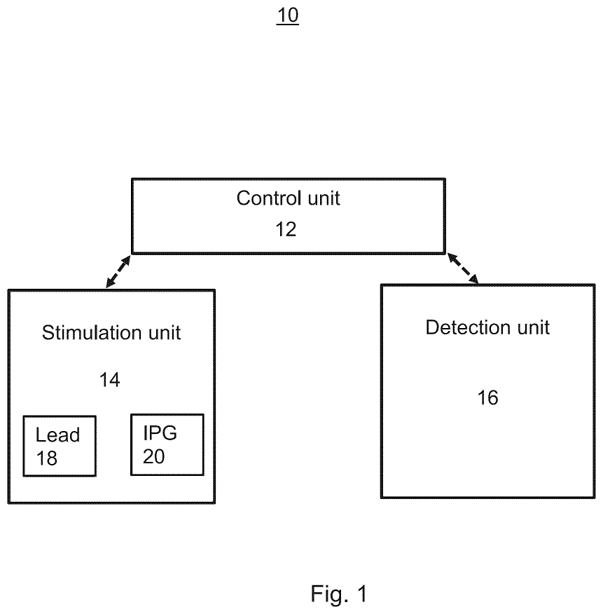

FIG. 1 shows a schematic overview of a neuromodulation/neurostimulation system 10 for the treatment of spasticity in a mammal according to an embodiment of the present invention.

In the shown embodiment, said mammal is a human affected by SCI and/or other neurological disorders such as a stroke, multiple sclerosis, cerebral palsy or cancer of the neurological tissue which impair operation of descending neurological pathways that normally facilitate motor control.

The system 10 includes at least one control unit 12.

In the shown embodiment, the system 10 includes a single control unit 12.

The control unit 12 is configured and arranged to provide stimulation data.

The system 10 further comprises at least one stimulation unit 14.

In the shown embodiment, the system 10 includes a single stimulation unit 14.

The stimulation unit 14 is operatively connected to the control unit 12.

The stimulation unit 14 is configured and arranged to deliver epidural electrical stimulation (EES) to the spinal cord of a subject according to said stimulation data, provided by the control unit 12.

The stimulation unit 14 includes a biocompatible implantable lead 18.

In particular, the implantable lead 18 is configured and arranged to cover at least a portion of the spinal cord of said subject to deliver EES to the dorsal roots innervating the spastic muscles to target neuronal circuitry responsible for spastic episodes.

Accordingly, optimally targeted stimulation can be delivered to reduce frequency and/or severity of spastic episodes in specific muscle groups while not influencing others, thereby preventing the occurrence unwanted side effects on normal muscular functioning.

Not shown is that the implantable lead 18 may include one or more biocompatible implantable multi-electrode arrays.

Not shown is that the one or more multi-electrode arrays may be used to deliver monopolar or multipolar stimulation.

Not shown is that the electrodes of the multi-electrode array may be transversally positioned with respect to the spinal cord of said subject.

Not shown is that, alternatively, the electrodes of the multi-electrode array may be arranged in two or more columns disposed in a longitudinal direction relative to the spinal cord.

The electrodes may be manufactured with materials and methods that are known in the art. Accordingly, description of said materials and manufacturing methods will be omitted for the sake of brevity.

In the shown embodiment, the stimulation unit 14 further includes an Implantable Pulse Generator (IPG) 20.

Advantageously, the implantable lead 18 is configured and arranged to deliver EES to the spinal cord of said mammal at spinal cord level responsible for the lower limbs, preferably at level T11/T12-L3 (vertebrae).

Additionally or alternatively, the implantable lead 18 may be configured and arranged to deliver EES to the spinal cord of said mammal at spinal cord level responsible for the upper limbs, preferably over the cervical region of the spinal cord.

Additionally or alternatively, the implantable lead 18 may be configured and arranged to deliver EES to the spinal cord of said mammal at spinal cord level responsible for the trunk muscles, in particular over the thoracic region of the spinal cord.

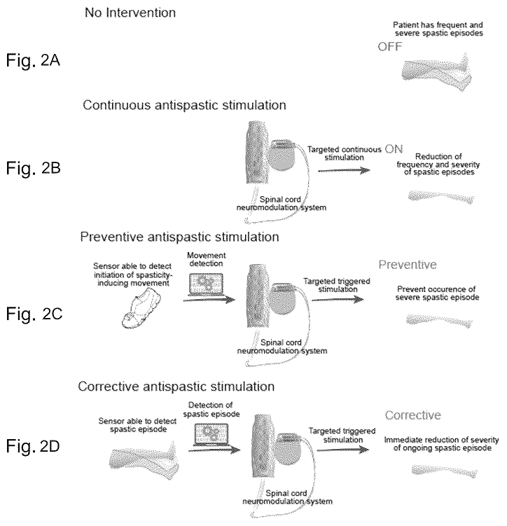

FIG. 2A shows a condition where no stimulation is delivered to a subject (e.g., a patient).

As illustrated in FIG. 2A; in the absence of stimulation, the subject shows frequent and severe spastic episodes.

FIG. 2B shows an embodiment where continuous EES is delivered to targeted locations of the spinal cord of a subject.

In this embodiment, the control unit 12 is configured and arranged to control the stimulation unit 12 to deliver continuous EES to the spinal cord of said subject.

As shown in FIG. 2B, by continuously delivering EES to targeted locations of the spinal cord, frequency of spastic events and severity of the spasms may be effectively reduced.

FIG. 2C shows an embodiment where EES is delivered to targeted locations of the spinal cord upon detecting onset of a spasticity-inducing activity and/or movement in a subject.

In this embodiment, the system 10 further includes a detection unit 16.

In this embodiment, the detection unit 16 is configured and arranged to detect onset of a spasticity-inducing activity and/or movement in said subject based on one or more preset parameters stored in a memory.

In this embodiment, the detection unit 16 is further configured and arranged to transmit an output signal to the control unit 12 when onset of spasticity-inducing activity and/or movement is detected.

In this embodiment, the control unit 12 is configured to operate the stimulation unit 14 to provide EES to the spinal cord of said subject in response to the received signal from the detection unit 16, to prevent occurrence of a spastic episode.

For instance, said spasticity-inducing activity and/or movement may include transferring of the subject from a wheelchair to bed.

As shown in FIG. 2C, by triggering targeted EES before a subject is performs an activity and/or movement which is identified as spasticity-inducing, it is possible to temporally prevent a spastic episode from occurring.

FIG. 2D shows an embodiment where EES is delivered to targeted location of the spinal cord upon detecting onset of spastic muscle activity in a subject.

In this embodiment, the system 10 further includes a detection unit 16.

In this embodiment, the detection unit 16 is configured and arranged to detect onset of spastic muscle activity in said subject.

In this embodiment, the detection unit 16 is further configured and arranged to transmit an output signal to the control unit 12 when onset of spastic muscle activity is detected.

In this embodiment, the control unit 12 is configured to operate the stimulation unit 14 to provide EES to the spinal cord of said subject in response to the received signal from the detection unit 16.

As shown in FIG. 2D, by triggering targeted EES when onset of spastic muscle activity is detected, severity of the ongoing spastic episode can be significantly reduced.

Not shown is that the detection unit 16 may include at least one electromyography sensor unit.

Not shown is that, either additionally or alternatively, the detection unit 16 may include at least one inertial measurement unit.

The stimulation data may comprise at least frequency, amplitude, pulse width, and/or anodic/cathodic configurations.

The optimization of stimulation data, such as frequency, amplitude and/or pulse width, plays a significant role in mitigating or preventing spastic episodes in a subject after SCI and/or other neurological disorders.

Advantageously, the stimulation frequency may be between 100 Hz and 1500 Hz.

Advantageously, the pulse width may be between 60 μs and 1000 μs.

Preferably, the pulse width may be of 300 μs.

Advantageously, the stimulation amplitude is around motor threshold level.

Not shown is that the system 10 may further include an oscillation control module.

In particular, the oscillation control module may be configured and arranged to provide an input between 0.01 Hz and 0.2 Hz low frequency oscillation in the amplitude and/or frequency.

Preferably, the oscillation control module may be configured and arranged to provide an input of 0.1 Hz low frequency oscillation in the amplitude and/or frequency.

Not shown is that the stimulation unit 14 may be configured and arranged to provide at least one burst train stimulation pulse.

Preferably, the stimulation unit 14 may be configured and arranged to provide at least one burst of several pulses.

More preferably, the stimulation unit 14 may be configured and arranged to provide at least one burst of 2 to 5 pulses.

The neuromodulation/neurostimulation system 10 according to the invention can be used for implementing a method for the treatment of spasticity in a mammal

In particular, in the present example, said mammal is a human affected by SCI and/or other neurological disorders such as a stroke, multiple sclerosis, cerebral palsy or cancer of neurological tissue which impair operation of descending neurological pathways that normally facilitate motor control.

The method may include:
positioning the biocompatible implantable lead 18 on the body of a subject to cover at least a portion of the spinal cord of said subject, and deliver EES to the dorsal roots innervating the spastic muscles to target neuronal circuitry responsible for spastic episodes, and
controlling the stimulation unit 14 to deliver EES to the spinal cord of said subject according to stimulation data provided by the control unit 12.

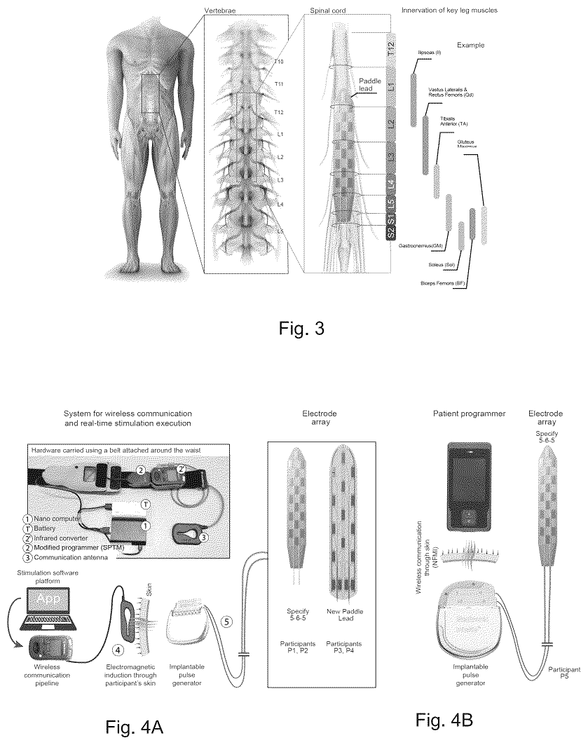

In one example, the method may include delivering EES to the spinal cord of said mammal at spinal cord level responsible for the lower limbs, preferably at level T11/T12-L3 (vertebrae) (or looking at spinal cord itself the section of the spinal cord and its dorsal roots for T12 to S2) (cf. also FIG. 3).

In one example, the method may include delivering EES to the spinal cord of said mammal at spinal cord level responsible for the upper limbs, preferably over the cervical region of the spinal cord.

In one example, the method may include delivering EES to the spinal cord of said mammal at spinal cord level responsible for the trunk muscles, in particular over the thoracic region of the spinal cord. In one example, the method may include delivering continuous EES to the spinal cord of said subject.

In this example, the method operates the system 10 as an open-loop system.

In one example, the method may include:
detecting, through the detection unit 16, onset of spastic muscle activity in said subject based on one or more preset parameters stored in a memory;
transmitting an output signal to the control unit 12 when onset of spastic muscle activity is detected, and
operating the stimulation unit 14 to provide EES to the spinal cord of said subject in response to the received signal from the detection unit 12.

In this example, the method operates the system 10 as a closed-loop system.

In one example, the method may include:
detecting, through the detection unit 12, onset of a spasticity-inducing activity and/or movement in a subject based on one or more preset parameters stored in a memory;
transmitting an output signal to the control unit 12 when a spasticity-inducing activity and/or movement is detected, and
operating the stimulation unit 14 to provide EES to the spinal cord of said subject in response to the received signal from the detection unit 12.

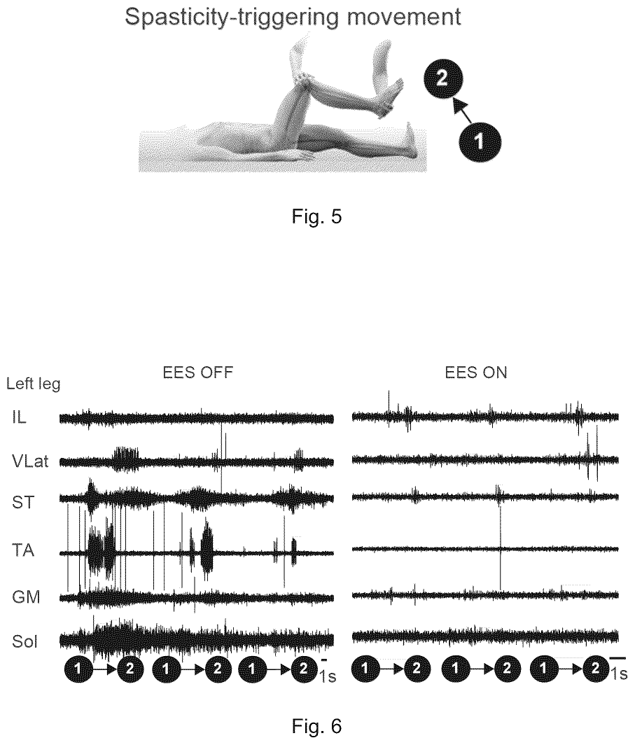

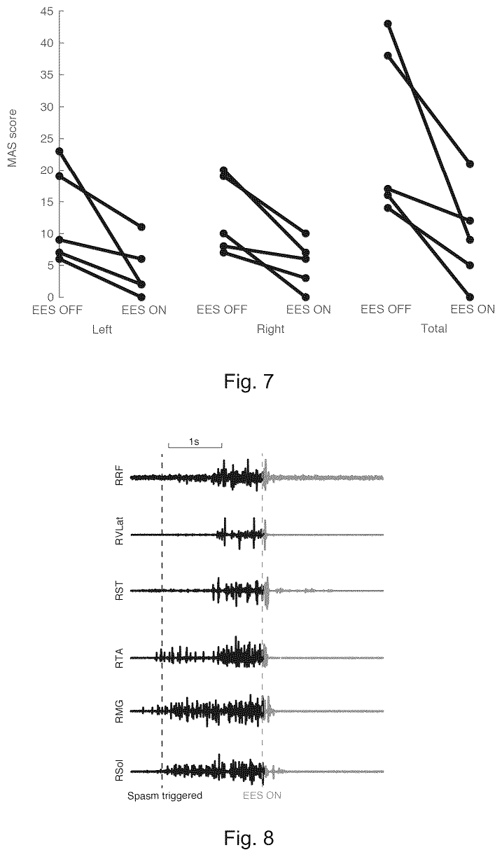

An example of a spasticity-triggering movement is shown in FIG. 5.

In this example, the method as well operates the system 10 as a closed-loop system.

Advantageously, the detection unit 16 may include at least one electromyography sensor unit.

Additionally or alternatively, the detection unit 16 may include at least one inertial measurement unit.

In one example, the method may include delivering EES with stimulation frequency between 100 Hz and 1500 Hz.

In one example, the method may include delivering EES with pulse width between 60 μs and 1000 μs.

In a preferred example, the method may include delivering EES with pulse width of 300 μs.

In one example, the method may include delivering EES with amplitude around the motor threshold level.

In one example the method may include providing, through an oscillation control module, an input between 0.01 Hz and 0.2 Hz low frequency oscillation in the amplitude and/or frequency.

Preferably, the method may include providing an input of 0.1 Hz low frequency oscillation in the amplitude and/or frequency.

The method may include providing at least one burst train stimulation pulse.

Preferably, the method may include providing at least one burst of several pulses.

More preferably, the method may include providing at least one burst of 2 to 5 pulses.

### Clinical Data

### Targeted EES for the Lower Limbs

An experimental concept of targeted EES for the lower limbs is shown in FIG. 3.

Leg muscles are innervated by the lumbosacral spinal cord in an individual-specific rostro-caudal spatial organization.

An electrode array (also called paddle lead) is implanted on the lumbosacral spinal cord of a patient to stimulate specific spinal segments innervating respective specific muscles.

### Clinical Studies and Implanted System

Anti-spastic stimulation was tested in multiple participants P1-P5 of the STIMO and STIMO-HEMO clinical trials (clinicaltrials.gov IDs NCT02936453 and NCT04994886).

All the involved participants P1-P5 presented chronic traumatic spinal cord injury with elevated levels of spasticity in the lower limbs.

Participants were implanted with a 16-electrode paddle lead on the lumbosacral spinal cord.

The specific stimulation systems used in the clinical study are shown in FIG. 4A and FIG. 4B.

In particular, participants P1, P2 and P5 received a commercially available Medtronic Specify 5-6-5 paddle lead used off-label, whereas participants P3 and P4 were implanted with a more targeted non-commercial implant from Onward medical.

For participants P1-P4, the paddle lead was connected to a commercially available IPG (ActivaRC, Medtronic) endowed with real-time triggering capacities through research firmware (FIG. 4A). This allowed custom-built software platforms to communicate with the IPG and trigger the correct stimulation at the correct location and time.

Participant P5 received a commercially available IPG (Intellis, Medtronic) used off-label (FIG. 4B).

### Anti-Spastic Effects of Targeted Spinal Cord Stimulation

FIG. 5 shows an example of a spasticity-inducing passive movement of a subject.

FIG. 6 shows the effect of delivery of anti-spastic targeted stimulation on the left leg of participant P2.

When stimulation is turned off (see the left side of FIG. 6), an episode of spasticity in the left leg is detected.

Delivery of targeted stimulation (see the right side of FIG. 6 during an ongoing spastic event dramatically reduces muscle spasticity.

Reduction in Modified Ashworth Scale (MAS) with Targeted EES

The Modified Ashworth Scale (MAS) is a widely used clinical method for measuring muscle spasticity.

MAS involves manual movement of a limb through its range of motion to passively stretch specific muscle groups. Patients are placed in a supine position and instructed to relax as much as possible. For testing flexor muscles, the specific joint for the respective muscle must be placed in a maximally flexed position and then be moved to a maximal extension over one second. The opposite applies for testing extensor muscles. Evaluation includes a six-point ordinal scale for grading the encountered resistance during passive muscle stretching (Bohannon and Smith, 1987).

In the present trial, the following muscles were tested for each participant P1-P5: Adductors, Iliopsoas, Gluteus Maximus, Biceps Femoris, Quadriceps, Tibialis Anterior, Gastrocnemius, Soleus, Toe flexors.

Here, to see the antispastic effect of the stimulation, the MAS measurement were performed in two different conditions. First, the MAS score was measured in a baseline condition, i.e. without stimulation. Second, EES was turned on with a targeted configuration and then muscle spasticity was measured in the same way as before.

The results of the trial for patients P1-P5 are shown in enclosed Table 1.

The obtained results showed that delivery of targeted EES highly reduced the MAS scores for all the involved participants P1-P5.

### Immediate Anti-Spastic Effects of Targeted Spinal Cord Stimulation

Targeted spinal cord stimulation can be turned on during an ongoing spastic episode.

It has been found that, in case targeted spinal cord stimulation is delivered during an ongoing spastic episode, the ongoing spastic activity is immediately reduced or even cancelled, and the muscles return to their baseline activity.

An example of the effect of delivery of targeted spinal cord stimulation during a spastic episode is shown in FIG. 8.

### Combination of Antispastic Stimulation and Antispastic Medication

Table 2 shows a comparison of the obtained effects of antispastic stimulation and antispastic medication on lower-limb in Patient 5 (MAS scores).

The results showed that antispastic EES alone is overall more efficient in reducing the effects of a spastic episode than administration of a medicament (dosage=10 mg of baclofen) alone.

Furthermore, the results demonstrated that delivery of antispastic EES and the administration of a medicament (dosage=10 mg of baclofen) act synergistically.

### REFERENCES

10 Neuromodulation/neurostimulation system
12 Control unit
14 Stimulation unit
16 Detection unit
18 Biocompatible implantable lead
20 Implantable Pulse Generator (IPG)

TABLE 2
Comparison of the effects of antispastic stimulation and
antispastic medication on lower-limb (MAS scores).

| Participant P5 | No medication | With medication | Difference |
|---|---|---|---|
| No stimulation | 22 | 16 | −6 |
| With | 2 | 0 | −2 |
| stimulation | | | |
| Difference | −20 | −16 | |

No intervention vs combined intervention (stimulation + medication): −22
Only medication vs only stimulation: −14

1. A neuromodulation/neurostimulation system for treatment of spasticity in a mammal, said system comprising:
at least one control unit configured and arranged to provide stimulation data, and
at least one stimulation unit, operatively connected to the at least one control unit, said at least one stimulation unit being configured and arranged to deliver epidural electrical stimulation to a spinal cord of said mammal, according to said stimulation data,
wherein the at least one stimulation unit includes a biocompatible implantable lead configured and arranged to cover at least a portion of the spinal cord of said mammal to deliver epidural electrical stimulation to dorsal roots innervating spastic muscles to target neuronal circuitry responsible for spastic episodes.

2. The neuromodulation/neurostimulation system according to claim 1, wherein the implantable lead is configured and arranged to deliver epidural stimulation to the spinal cord of said mammal
at a spinal cord level responsible for lower limbs, upper limbs, and/or trunk muscles.

3. The neuromodulation/neurostimulation system according to claim 1, wherein the at least one control unit is configured and arranged to control the at least one stimulation unit to deliver continuous epidural electrical stimulation to the spinal cord of said mammal.

4. The neuromodulation/neurostimulation system according to claim 1, further comprising a detection unit,
wherein the detection unit is configured and arranged to:
detect onset of spastic muscle activity in said mammal, and
when onset of spastic muscle activity is detected, transmit an output signal to the at least one control unit,
wherein the at least one control unit is configured to operate the at least one stimulation unit to provide epidural electrical stimulation to the spinal cord of said mammal in response to the received signal from the detection unit.

5. The neuromodulation/neurostimulation system according to claim 1, further comprising a detection unit,
wherein the detection unit is configured and arranged to:
detect onset of a spasticity-inducing activity and/or movement in said mammal based on one or more preset parameters stored in a memory, and
when onset of spasticity-inducing activity and/or movement is detected, transmit an output signal to the at least one control unit,
wherein the at least one control unit is configured to operate the at least one stimulation unit to provide epidural electrical stimulation to the spinal cord of said mammal in response to the received signal from the detection unit, to prevent occurrence of a spastic episode.

6. The neuromodulation/neurostimulation system according to claim 4, wherein the detection unit includes at least one electromyography sensor unit and/or at least one inertial measurement unit.

7. The neuromodulation/neurostimulation system according to claim 1, wherein the stimulation data comprise at least frequency, amplitude, pulse width and/or anodic/cathodic configurations.

8. The neuromodulation/neurostimulation system according to claim 7, wherein the at least one control unit comprises an oscillation control module, wherein the oscillation control module is configured and arranged to provide an input between 0.01 Hz and 0.2 Hz low frequency oscillation in the amplitude and/or the frequency.

9. The neuromodulation/neurostimulation system according to claim 1, wherein the at least one stimulation unit is configured and arranged to provide at least one burst train stimulation pulse.

10. The neuromodulation/neurostimulation system according to claim 9 wherein the at least one stimulation unit is configured and arranged to provide at least one burst of several pulses.

11. The neuromodulation/neurostimulation system according to claim 1, wherein the implantable lead includes one or more biocompatible implantable multi-electrode arrays.

12. The neuromodulation/neurostimulation system according to claim 1, wherein the at least one stimulation unit includes an Implantable Pulse Generator.

13. The neuromodulation/neurostimulation system according to claim 1, wherein said mammal is a human affected by spinal cord injury and/or other neurological disorders.

14. A use of the neuromodulation/neurostimulation system according to claim 1 for the treatment of spasticity in a mammal.

15. The neuromodulation/neurostimulation system according to claim 2, wherein the implantable lead is configured and arranged to deliver epidural stimulation to the spinal cord of said mammal at the spinal cord level responsible for the lower limbs at level T11/T12-L3 (vertebrae).

16. The neuromodulation/neurostimulation system according to claim 2, wherein the implantable lead is configured and arranged to deliver epidural stimulation to the spinal cord of said mammal at the spinal cord level responsible for the upper limbs over a cervical region of the spinal cord.

17. The neuromodulation/neurostimulation system according to claim 2, wherein the implantable lead is configured and arranged to deliver epidural stimulation to the spinal cord of said mammal at the spinal cord level responsible for the trunk muscles over a thoracic region of the spinal cord.

18. The neuromodulation/neurostimulation system according to claim 7, wherein the frequency is between 100 Hz and 1500 Hz, the pulse width is between 60 μs and 1000 μs, and the amplitude is around motor threshold level.

19. The neuromodulation/neurostimulation system according to claim 10, wherein the at least one burst of several pulses is 2 to 5 pulses.

20. The use of claim 14, wherein the mammal is a human.
